(19) United States
(12) Patent Application Publication
(10) Pub. No.: US 2021/0134445 A1
(43) Pub. Date: May 6, 2021

(54) METHODS AND SYSTEMS FOR FACILITATING MANAGING FACILITIES FOR PATIENTS

(71) Applicants: Sunil Ramesh Pandya, Naples, FL (US); John Alan Gardner, wellington, FL (US)

(72) Inventors: Sunil Ramesh Pandya, Naples, FL (US); John Alan Gardner, wellington, FL (US)

(73) Assignee: transitional care strategies LLC, Miami Beach, FL (US)

(21) Appl. No.: 17/085,013

(22) Filed: Oct. 30, 2020

Related U.S. Application Data
(60) Provisional application No. 62/927,990, filed on Oct. 30, 2019.

(51) Int. Cl. G16H 40/20; G16H 10/60; G16H 50/70; G16H 50/30; H04L 29/08; G08B 21/18

(52) U.S. Cl. CPC G16H 40/20; G16H 10/60; G16H 50/70; G16H 50/20; H04L 67/12; G08B 21/182; G16H 50/30

(57) ABSTRACT

Disclosed herein is a method of facilitating managing facilities for patients, in accordance with some embodiments. Accordingly, the method comprises receiving an identifier from a first device, analyzing the identifier, retrieving clinical data and patient data based on the analyzing, analyzing the clinical data and the patient data, determining a patient characteristic based on the analyzing of the clinical data and the patient data, identifying a second facility based on the determining, transmitting the second facility to a second device, initiating a timer based on the transmitting, generating an indication corresponding to a response receivable from the second device prior to the expiring of the timer, determining a primary second facility of the second facility based on the generating of the indication, generating referral data of the patient based on the determining of the primary second facility, and transmitting the referral data to a primary third device.

### FIELD OF THE INVENTION

Generally, the present disclosure relates to the field of data processing. More specifically, the present disclosure relates to methods and systems for facilitating managing facilities for patients.

### BACKGROUND OF THE INVENTION

Hospitals incur higher operating costs the longer a patient stays and Medicare incentivizes shorter Length of Stay metrics. Discharge planning is quite inefficient with case managers meeting daily with clinicians to get updates on patient status and then making phone calls to find availability at appropriate post-acute care facilities and arranging placement with the facility and the families of the patient. This can delay the discharge of the patient after the patient is medically ready to leave the hospital, costing the hospital money.

There may be other facility matching software on the market, but it does not have the ability to integrate with the clinical data in the EHR. The data has to be collected and entered by the case manager, which provides very little increased inefficiency. Matches can only be made once a case manager has collected and entered all of the data by meeting personally with clinicians and then entered the data manually.

Further, increased efficiency should reduce case management labor costs to the hospital and help clinicians spend more time treating patients.

Therefore, there is a need for improved methods and systems for facilitating managing facilities for patients that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method of facilitating managing facilities for patients, in accordance with some embodiments. Accordingly, the method may include receiving, using a communication device, at least one identifier associated with a patient of at least one patient from at least one first device. Further, the method may include analyzing, using a processing device, the at least one identifier. Further, the method may include retrieving, using a storage device, clinical data and patient data associated with the patient from an electronic health record associated with at least one first facility based on the analyzing of the at least one identifier. Further, the patient may be associated with the at least one first facility. Further, the method may include analyzing, using the processing device, the clinical data and the patient data. Further, the method may include determining, using the processing device, at least one patient characteristic associated with the patient based on the analyzing of the clinical data and the patient data. Further, the method may include identifying, using the processing device, at least one second facility based on the determining of the at least one patient characteristic. Further, the method may include transmitting, using the communication device, the at least one second facility to at least one second device. Further, the method may include initiating, using the processing device, a timer based on the transmitting of the at least one second facility. Further, the timer may be configured for expiring after at least one predetermined duration. Further, the method may include generating, using the processing device, an indication corresponding to a response receivable from the at least one second device prior to the expiring of the timer. Further, the method may include determining, using the processing device, at least one primary second facility of the at least one second facility based on the generating of the indication. Further, the method may include generating, using the processing device, referral data of the patient based on the determining of the at least one primary second facility. Further, the method may include transmitting, using the communication device, the referral data to at least one primary third device associated with the at least one primary second facility.

Further disclosed herein is a system for facilitating managing facilities for patients, in accordance with some embodiments. Accordingly, the system may include a communication device configured for receiving at least one identifier associated with a patient of at least one patient from at least one first device. Further, the communication device may be configured for transmitting at least one second facility to at least one second device. Further, the communication device may be configured for transmitting referral data to at least one primary third device associated with at least one primary second facility. Further, the system may include a storage device configured for retrieving clinical data and patient data associated with the patient from an electronic health record associated with at least one first facility based on the analyzing of the at least one identifier. Further, the patient may be associated with the at least one first facility. Further, the system may include a processing device communicatively coupled with the communication device and the storage device. Further, the processing device may be configured for analyzing the at least one identifier. Further, the processing device may be configured for analyzing the clinical data and the patient data. Further, the processing device may be configured for determining at least one patient characteristic associated with the patient based on the analyzing of the clinical data and the patient data. Further, the processing device may be configured for identifying the at least one second facility based on the determining of the at least one patient characteristic. Further, the processing device may be configured for initiating a timer based on the transmitting of the at least one second facility. Further, the timer may be configured for expiring after at least one predetermined duration. Further, the processing device may be configured for generating an indication corresponding to a response receivable from the at least one second device prior to the expiring of the timer. Further, the processing device may be configured for determining the at least one primary second facility of the at least one second facility based on the generating of the indication. Further, the processing device may be configured for generating the referral data of the patient based on the determining of the at least one primary second facility.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating managing facilities for patients, in accordance with some embodiments.

FIG. 3 is a flowchart of a method for facilitating managing facilities for patients, in accordance with some embodiments.

FIG. 4 is a flowchart of a method for generating at least one alert, in accordance with some embodiments.

FIG. 5 is a flowchart of a method for transmitting the at least one decision maker information to the at least one first device, in accordance with some embodiments.

FIG. 6 is a flowchart of a method for transmitting the at least one second facility to the at least one first device, in accordance with some embodiments.

FIG. 7 is a flowchart of a method for generating at least one facility characteristic, in accordance with some embodiments.

FIG. 8 is a flowchart of a method for matching the at least one patient characteristic with the at least one facility characteristic, in accordance with some embodiments.

FIG. 9 is a flowchart of a method for generating a score, in accordance with some embodiments.

FIG. 10 is a flowchart of a method for generating at least one status, in accordance with some embodiments.

FIG. 11 is a flowchart of a method for generating a notification, in accordance with some embodiments.

FIG. 12 is a flowchart of a method to facilitate hospital dispatch planning, in accordance with some embodiments.

FIG. 13 shows an architecture overview associated with the system, in accordance with some embodiments.

FIG. 14 shows an architecture overview, in accordance with some embodiments.

FIG. 15 is a flow diagram of a method for facility matching, in accordance with some embodiments.

FIG. 16 is a flow diagram of a method for assigning patient bed categories, in accordance with some embodiments.

FIG. 17 shows a DataBase (DB) schema associated with the system, in accordance with some embodiments.

FIG. 18 shows a DB schema associated with the system, in accordance with some embodiments.

FIG. 19 shows a DB schema associated with the system, in accordance with some embodiments.

FIG. 20 shows a DB schema associated with the system, in accordance with some embodiments.

FIG. 21 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for facilitating managing facilities for patients, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure describes methods and systems for facilitating managing facilities for patients. Accordingly, a hospital case manager may use a Case Manager application to select a patient that may need some sort of post-acute care placement. Further, the case manager application may then pull patient ID data, insurance data, and the patient's social information and may ask the case manager to confirm that all of the information may be correct before proceeding. After the information may be verified, clinical information from the health record, including information contained within the progress notes of the treating physician, physical therapists, occupational therapists, and speech therapists. The entirety of information is then processed by our matching algorithm to find all of the participating post-acute care facilities in the area which meet the patient's requirements and also accept their insurance. Further, all of the potential matches from the participating facilities are then made available to the patient/family member/medical decision-maker via web application with descriptions/photos of each facility. The application allows them to select a facility from the application or schedule a visit first. Further, a timer starts as soon as matches were made available to the decision-maker. After a set amount of time, the application indicates to the case manager that a decision has not been reached yet, and the manual follow up may be needed. At this stage, the application provides the decision-maker contact info (phone and e-mail) for quick access. After another predetermined time period elapses, a more urgent call for follow up is made to the case manager. If the family/decision-maker is non-responsive or not participating, the case managers can make the selection themselves. Further, once a selection has been made, the treating physician is notified via SMS that their patient has been matched to the selected facility and the referral for the facility is generated and sent to the facility agent application. Further, the application tracks time from admission to discharge as well as time spent at each step to provide the hospital administration with a length of stay and utilization data.

According to some embodiments, a placement algorithm based on the medical acuity of the patient, the patient's need for various rehabilitation and or skilled nursing services, and various requirements and exclusions associated with the various possible discharge dispositions are disclosed.

According to some embodiments, a feature to allow facilities to divide bed availability by category is provided. For example, general beds, isolation beds, Medicaid beds, Memory unit beds, and charity beds. The requirements of each patient are checked against the current availability of each facility before being considered a potential match.

According to some embodiments, a feature that allows the post-acute care facilities to set a treatment plan cost threshold for possible acceptance is disclosed. The disclosed application will calculate the facility cost of the proposed treatment plan and consider the facility as a potential match if the calculated cost is below the threshold set by the facility. However, this facility acceptance process is more complicated than anticipated, and that has been the delay in completing the placement algorithm. We have come up with a process change as a workaround for the difficulty we are having implementing this feature to get to market, but plan on incorporating this feature in the next version.

According to some embodiments, a method for automated insurance verification is disclosed.

Further, the disclosed system may facilitate hospital discharge planning more efficient by pulling patient information and clinical information from Electronic Medical Record (EMR), processing that information through a matching algorithm, and sending all potential matched discharge disposition facilities to the patient's family via an app for approval.

Further, hospitals incur higher operating costs the longer a patient stays and Medicare incentivizes shorter Length of Stay metrics. Discharge planning is quite inefficient with case managers meeting daily with clinicians to get updates on patient status than making phone calls to find availability at appropriate post-acute care facilities and arranging placement with the facility and the families. This can delay the discharge of the patient after the patient is medically ready to leave the hospital, costing the hospital money.

There is other facility matching software on the market, but it does not have the ability to integrate with the clinical data in Electronic Health Record (EHR) or the EMR. The data has to be collected and entered by the case manager, which provides very little increase in efficiency. Matches can only be made once a case manager has collected and entered all of the data by meeting personally with clinicians and then entered the data manually.

By mining the relevant data from the EHR, the disclosed system may save the case managers and the clinicians time, making both more efficient. The increased efficiency should reduce case management labor costs to the hospital and help clinicians spend more time treating patients. Further, Electronic Medical Record integration facilitates the gathering of relevant information, improving the efficiency of both case managers and clinicians. Further, the matching algorithm processes data to match patients with appropriate local facilities. Further, an integrated family/decision-maker app allows patient's decision-makers to view appropriate matches with full descriptions so that they can make an informed choice of facility for their loved ones. Further, the disclosed system may be configured for receiving real-time information on the availability of participating post-acute care facilities. Further, the disclosed system may be configured for gathering patient data and clinical data from the Electronic Medical Record, including patient ID information, insurance information, social information, physician progress notes and speech therapy, occupational therapy, and physical therapy progress notes. Further, the disclosed system may be configured for processing the patient data and the clinical data into an algorithm that determines which local facilities meet the patient's needed level of acuity (level of services required depending on how sick they are and how much assistance they need). Further, the disclosed system may be configured for discharge planning. Further, the disclosed system may be configured for Electronic Medical Record integration. Further, the disclosed system may be configured for discharge disposition matching. Further, the disclosed system may be configured for post-acute care facility matching. Further, the disclosed system may include an integrated patient family/decision-maker web-based application. Further, the disclosed system may be configured for real-time post-acute care facility availability status tracking.

The disclosed method may include a hospital case manager using the disclosed application (or app) Case Manager (an exemplary embodiment of the disclosed system) to select a patient that will need some sort of post-acute care placement. The Case Manager app may pull patient ID data, insurance data, and the patient's social information and asks the case manager to confirm that all of the information is correct before proceeding. Further, after the information is verified, clinical information from the health record, including information contained within the progress notes of the treating physician, physical therapists, occupational therapists, and speech therapists. The entirety of information is then processed by a matching algorithm associated with the disclosed system to find all of the participating post-acute care facilities in the area which meet the patient's requirements and also accept their insurance. Further, all of the potential matches from the participating facilities are then made available to the patient/family member/medical decision-maker via a web app with descriptions/photos of each facility. The Case Manager app allows them to select a facility from the Case Manager app or schedule a visit first. Further, a timer starts as soon as matches were made available to the decision-maker. After a set amount of time, the app indicates to the case manager that a decision has not been reached yet, and the manual follow up may be needed. At this stage, the Case Manager app provides the decision-maker contact info (phone and e-mail) for quick access. After another predetermined time period elapses, a more urgent call for follow up is made to the case manager. If the family/decision-maker is non-responsive or not participating, the case managers can make the selection themselves. Once a selection has been made, the treating physician is notified via SMS that their patient has been matched to the selected facility and the referral for the facility is generated and sent to the facility agent app. The app tracks time from admission to discharge as well as time spent at each step to provide the hospital administration with the length of stay and utilization data.

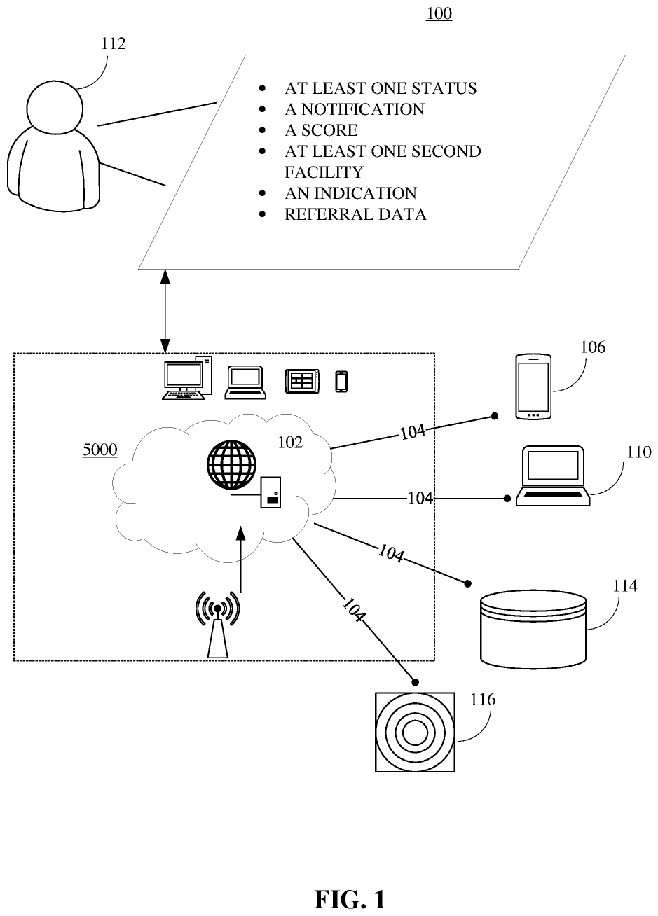

Referring now to figures, FIG. 1 is an illustration of an online platform 100 consistent with various embodiments of the present disclosure. By way of non-limiting example, the online platform 100 for facilitating managing facilities for patients may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 2100.

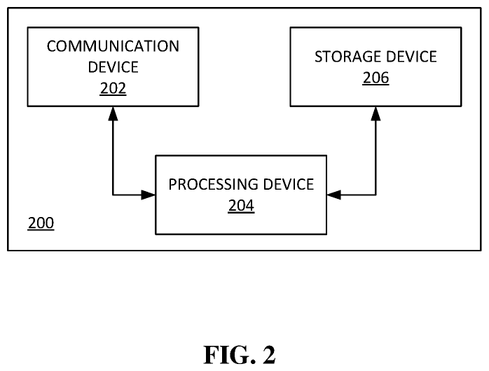

FIG. 2 is a block diagram of a system 200 for facilitating managing facilities for patients, in accordance with some embodiments. Accordingly, the system 200 may include a communication device 202, a processing device 204, and a storage device 206.

Further, the communication device 202 may be configured for receiving at least one identifier associated with a patient of at least one patient from at least one first device. Further, the communication device 202 may be configured for transmitting at least one second facility to at least one second device. Further, the communication device 202 may be configured for transmitting referral data to at least one primary third device associated with at least one primary second facility. Further, the at least one identifier may include a name, a number, an image, etc. Further, the at least one identifier may be unique to the patient. Further, the patient may include at least one individual. Further, the at least one first device may include a computing device such as a smartphone, a laptop, a tablet, a desktop, a smartwatch, and so on. Further, the at least one second device may include a computing device such as a smartphone, a laptop, a tablet, a desktop, a smartwatch, and so on. Further, the at least one third device may include a computing device such as a smartphone, a laptop, a tablet, a desktop, a smartwatch, and so on.

Further, the storage device 206 may be configured for retrieving clinical data and patient data associated with the patient from an electronic health record (EHR) associated with at least one first facility based on the analyzing of the at least one identifier. Further, the patient may be associated with the at least one first facility. Further, the at least one first facility may include at least one medical facility. Further, the at least one medical facility may include a hospital, a clinic, etc.

Further, the processing device 204 may be communicatively coupled with the communication device 202 and the storage device 206. Further, the processing device 204 may be configured for analyzing the at least one identifier. Further, the processing device 204 may be configured for analyzing the clinical data and the patient data. Further, the processing device 204 may be configured for determining at least one patient characteristic associated with the patient based on the analyzing of the clinical data and the patient data. Further, the processing device 204 may be configured for identifying the at least one second facility based on the determining of the at least one patient characteristic. Further, the processing device 204 may be configured for initiating a timer based on the transmitting of the at least one second facility. Further, the timer may be configured for expiring after at least one predetermined duration. Further, the processing device 204 may be configured for generating an indication corresponding to a response receivable from the at least one second device prior to the expiring of the timer. Further, the processing device 204 may be configured for determining the at least one primary second facility of the at least one second facility based on the generating of the indication. Further, the processing device 204 may be configured for generating the referral data of the patient based on the determining of the at least one primary second facility. Further, the analyzing may include identifying the patient. Further, the at least one patient characteristic may include at least one care requirement, at least one personal preference, at least one diagnosis, at least one patient address, etc. Further, the at least one second facility may include at least one care facility. Further, the at least one care facility may include at least one rehabilitation center, etc. Further, the timer may be configurable for pausing based on the response. Further, the pausing prevents the expiring of the timer.

Further, in some embodiments, the timer may be configurable for pausing prior to the expiring based on the response. Further, the pausing of the timer prevents the expiring of the timer.

Further, in some embodiments, the processing device 204 may be further configured for generating at least one alert subsequent to the expiring of the timer. Further, the communication device 202 may be further configured for transmitting the at least one alert to the at least one first device.

Further, in some embodiments, the storage device 206 may be further configured for retrieving at least one decision maker information associated with at least one decision maker subsequent to the expiring of the timer. Further, the communication device 202 may be further configured for transmitting the at least one decision maker information to the at least one first device.

Further, in some embodiments, the processing device 204 may be further configured for initiating a first timer based on the transmitting of the at least one decision maker information. Further, the first timer may be configured for expiring after at least one first predetermined duration. Further, the communication device 202 may be configured for transmitting the at least one second facility to the at least one first device subsequent to the expiring of the first timer. Further, the communication device 202 may be configured for receiving at least one primary second facility indication corresponding to the at least one primary second facility from the at least one first device. Further, the determining of the at least one primary second facility of the at least one second facility may be based on the receiving of the at least one primary second facility indication.

Further, in some embodiments, the communication device 202 may be further configured for receiving at least one second facility information from at least one third device. Further, the processing device 204 may be configured for analyzing the at least one second facility information based on at least one predetermined criterion. Further, the processing device 204 may be configured for generating at least one facility characteristic of each second facility of the at least one second facility based on the analyzing of the at least one second facility information. Further, the storage device 206 may be further configured for storing the at least one facility characteristic and the at least one second facility information. Further, the at least one facility characteristic may include at least care service, at least one address, etc.

Further, in some embodiments, the storage device 206 may be further configured for retrieving the at least one facility characteristic associated with the each second facility. Further, the processing device 204 may be further configured for matching the at least one patient characteristic with the at least one facility characteristic based on the retrieving of the at least one facility characteristic. Further, the identifying of the at least one primary second facility may be based on the matching.

Further, in some embodiments, each patient characteristic of the at least one patient characteristic may include a predetermined value. Further, the processing device 204 may be configured for identifying at least one first patient characteristic of the at least one patient characteristic for the each second facility of the at least one second facility based on the matching. Further, the processing device 204 may be configured for generating a score of the each second facility based on the identifying of the at least one first patient characteristic. Further, the score may include the predetermined value for each first patient characteristic of the at least one first patient characteristic. Further, the identifying of the at least one primary second facility may be based on the generating of the score.

Further, in some embodiments, the processing device 204 may be configured for generating at least one status of the patient based on the analyzing of the patient data and the clinical data. Further, the identifying of the at least one second facility may be based on the generating of the at least one status. Further, the communication device 202 may be further configured for transmitting the at least one status to the at least one first device.

Further, in some embodiments, the communication device 202 may be configured for receiving at least one response from the at least one primary third device based on the transmitting of the referral data. Further, the communication device 202 may be configured for transmitting a notification to at least one of the at least one second device, the at least one first device, and at least one device of the at least one primary third device. Further, the at least one device may be associated with at least one facility. Further, the processing device 204 may be configured for identifying the at least one facility of the at least one primary second facility. Further, the processing device 204 may be configured for generating the notification based on the identifying of the at least one facility.

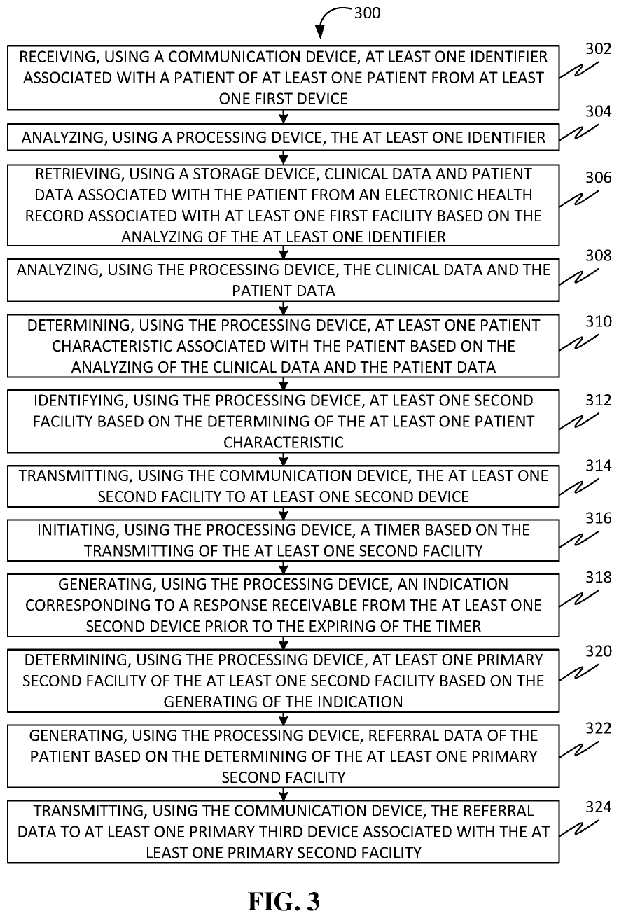

FIG. 3 is a flowchart of a method 300 for facilitating managing facilities for patients, in accordance with some embodiments. Accordingly, at 302, the method 300 may include receiving, using a communication device, at least one identifier associated with a patient of at least one patient from at least one first device.

Further, at 304, the method 300 may include analyzing, using a processing device, the at least one identifier.

Further, at 306, the method 300 may include retrieving, using a storage device, clinical data and patient data associated with the patient from an electronic health record associated with at least one first facility based on the analyzing of the at least one identifier. Further, the patient may be associated with the at least one first facility. Further, the at least one identifier may include a name, a number, an image, etc. Further, the patient may include at least one individual. Further, the analyzing may include identifying the patient. Further, the at least one first facility may include at least one medical facility. Further, the at least one medical facility may include a hospital, a clinic, etc.

Further, at 308, the method 300 may include analyzing, using the processing device, the clinical data and the patient data.

Further, at 310, the method 300 may include determining, using the processing device, at least one patient characteristic associated with the patient based on the analyzing of the clinical data and the patient data. Further, the at least one patient characteristic may include at least one care requirement, at least one personal preference, at least one diagnosis, at least one patient address, etc.

Further, at 312, the method 300 may include identifying, using the processing device, at least one second facility based on the determining of the at least one patient characteristic. Further, the at least one second facility may include at least one care facility. Further, the at least one care facility may include at least one rehabilitation center, etc.

Further, at 314, the method 300 may include transmitting, using the communication device, the at least one second facility to at least one second device.

Further, at 316, the method 300 may include initiating, using the processing device, a timer based on the transmitting of the at least one second facility. Further, the timer may be configured for expiring after at least one predetermined duration.

Further, at 318, the method 300 may include generating, using the processing device, an indication corresponding to a response receivable from the at least one second device prior to the expiring of the timer. Further, the timer may be configurable for pausing based on the response. Further, the pausing prevents the expiring of the timer. Further, the response may include an acknowledgement, a second facility indication, etc.

Further, at 320, the method 300 may include determining, using the processing device, at least one primary second facility of the at least one second facility based on the generating of the indication.

Further, at 322, the method 300 may include generating, using the processing device, referral data of the patient based on the determining of the at least one primary second facility.

Further at 324, the method 300 may include transmitting, using the communication device, the referral data to at least one primary third device associated with the at least one primary second facility.

Further, in some embodiments, the timer may be configurable for pausing prior to the expiring based on the response. Further, the pausing of the timer prevents the expiring of the timer.

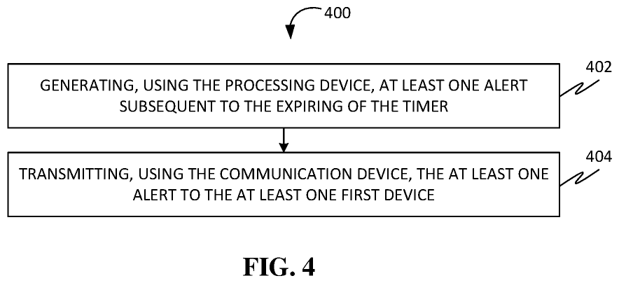

FIG. 4 is a flowchart of a method 400 for generating at least one alert, in accordance with some embodiments. Accordingly, at 402, the method 400 may include generating, using the processing device, at least one alert subsequent to the expiring of the timer.

Further, at 404, the method 400 may include transmitting, using the communication device, the at least one alert to the at least one first device.

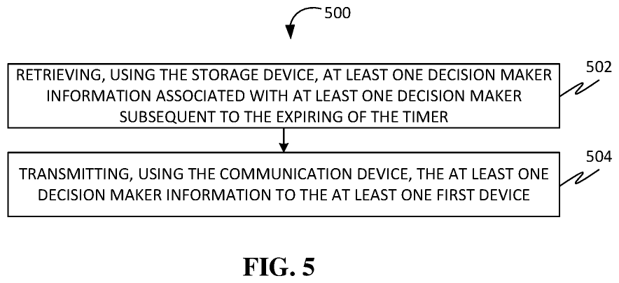

FIG. 5 is a flowchart of a method 500 for transmitting the at least one decision maker information to the at least one first device, in accordance with some embodiments. Accordingly, at 502, the method 500 may include retrieving, using the storage device, at least one decision maker information associated with at least one decision maker subsequent to the expiring of the timer.

Further, at 504, the method 500 may include transmitting, using the communication device, the at least one decision maker information to the at least one first device.

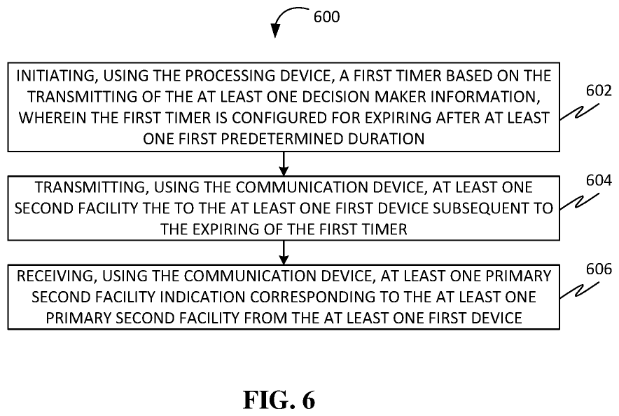

FIG. 6 is a flowchart of a method 600 for transmitting the at least one second facility to the at least one first device, in accordance with some embodiments. Accordingly, at 602, the method 600 may include initiating, using the processing device, a first timer based on the transmitting of the at least one decision maker information. Further, the first timer may be configured for expiring after at least one first predetermined duration.

Further, at 604, the method 600 may include transmitting, using the communication device, the at least one second facility to the at least one first device subsequent to the expiring of the first timer.

Further, at 606, the method 600 may include receiving, using the communication device, at least one primary second facility indication corresponding to the at least one primary second facility from the at least one first device. Further, the determining of the at least one primary second facility of the at least one second facility may be based on the receiving of the at least one primary second facility indication.

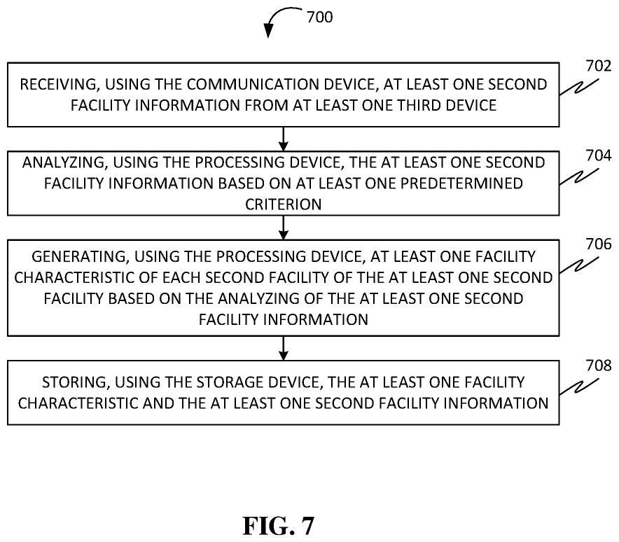

FIG. 7 is a flowchart of a method 700 for generating at least one facility characteristic, in accordance with some embodiments. Accordingly, at 702, the method 700 may include receiving, using the communication device, at least one second facility information from at least one third device.

Further, at 704, the method 700 may include analyzing, using the processing device, the at least one second facility information based on at least one predetermined criterion.

Further, at 706, the method 700 may include generating, using the processing device, at least one facility characteristic of each second facility of the at least one second facility based on the analyzing of the at least one second facility information. Further, the at least one facility characteristic may include at least one care service, at least one address, etc.

Further, at 708, the method 700 may include storing, using the storage device, the at least one facility characteristic and the at least one second facility information.

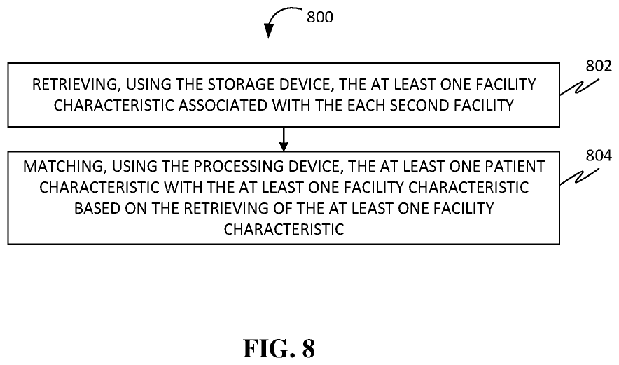

FIG. 8 is a flowchart of a method 800 for matching the at least one patient characteristic with the at least one facility characteristic, in accordance with some embodiments. Accordingly, at 802, the method 800 may include retrieving, using the storage device, the at least one facility characteristic associated with the each second facility.

Further, at 804, the method 800 may include matching, using the processing device, the at least one patient characteristic with the at least one facility characteristic based on the retrieving of the at least one facility characteristic. Further, the identifying of the at least one primary second facility may be based on the matching. Further, the matching may be based on a matching algorithm. Further, the matching algorithm may include a facility matching algorithm described below.

Below is a facility matching algorithm (Algorithm Filtering Stages) in accordance with some embodiments.

Location

Treatment Plan
Facility Type Determiners
   1. Medical Acuity Score (MMS)
      [1: HH, HHR, SNF, IRF, LTAC]
      [2: HHR, SNF, IRF, LTAC]
      [3: SNF, IRF, LTAC]
      [4: IRF, LTAC]
      [5: LTAC]
   2. Rehabilitation hours per week
      [null: don't filter]
      [n=0: don't filter]
      [n>0: [symbol] ]
      [<15: [symbol] ]
      [<10: [symbol] ]
   3. IV Therapy
      [null: don't filter]
      [‘antibio: don't filter]
      [‘blood_prod’: [symbol] ]
      [‘ppn’: [symbol] ]
      [‘cont_inf’: [symbol] F]
      [‘cont_inf_var’: [symbol] ]
      [‘chemo’: [symbol] ]
   4. Orthopedic number of join replacements
      [null or 1: don't filter]
      [>2: [symbol] ]
   5. CVL
      [true: [symbol] ]
   6. Ventilator
      [true: [symbol] ]
Individual Facility Determiners
   1. Bed Category (prepare structure to support multiple bed categories)
      [‘bed_category’=‘contact_isolation’, ‘available’=true]
      [‘bed_category’=‘memory_unit’, ‘available’=true]
      [‘bed_category’=‘medicaid’, ‘available’=true]
      [‘bed_category’=‘hmo’, ‘available’=true]
   2. Number of dialysis per week
      [‘dialysis_count’≥n]
SNF Days
   1. Visited in last 60 days
      [true, false]
   2. If above condition is TRUE, then check number of days left
      [0 . . . 100]

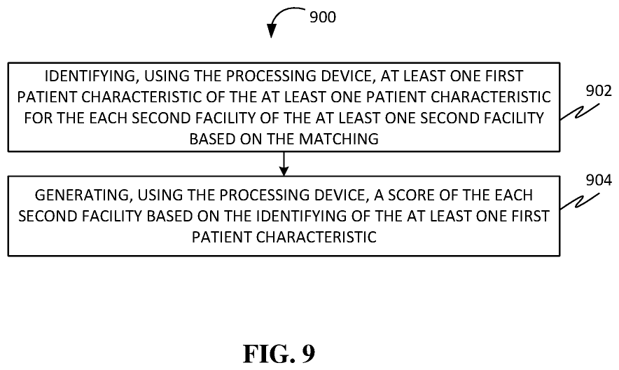

FIG. 9 is a flowchart of a method 900 for generating a score, in accordance with some embodiments. Accordingly, each patient characteristic of the at least one patient characteristic may include a predetermined value. Further, at 902, the method 900 may include identifying, using the processing device, at least one first patient characteristic of the at least one patient characteristic for the each second facility of the at least one second facility based on the matching.

Further, at 904, the method 900 may include generating, using the processing device, a score of the each second facility based on the identifying of the at least one first patient characteristic. Further, the score may include the predetermined value for each first patient characteristic of the at least one first patient characteristic. Further, the identifying of the at least one primary second facility may be based on the generating of the score.

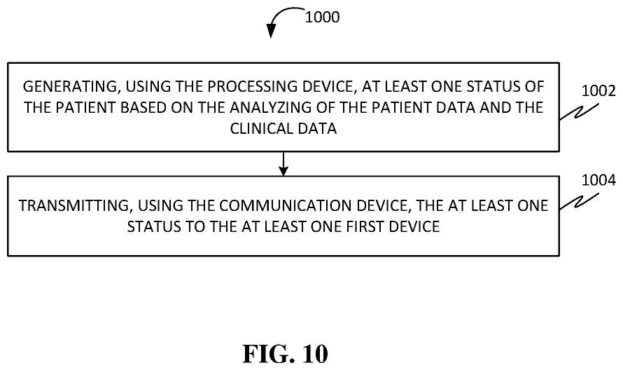

FIG. 10 is a flowchart of a method 1000 for generating at least one status, in accordance with some embodiments. Accordingly, at 1002, the method 1000 may include generating, using the processing device, at least one status of the patient based on the analyzing of the patient data and the clinical data. Further, the identifying of the at least one second facility may be based on the generating of the at least one status.

Further, at 1004, the method 1000 may include transmitting, using the communication device, the at least one status to the at least one first device.

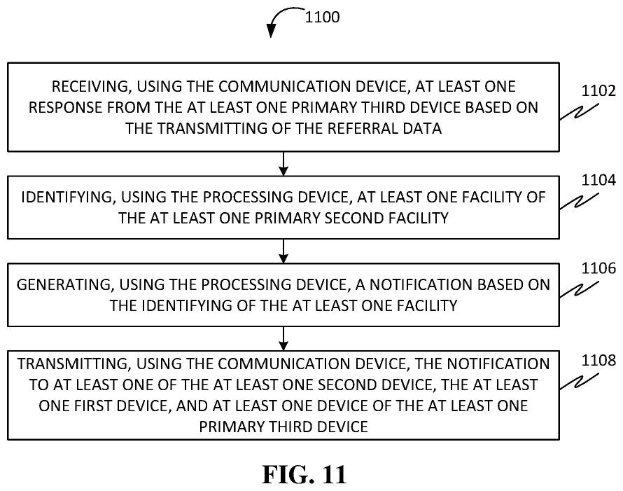

FIG. 11 is a flowchart of a method 1100 for generating a notification, in accordance with some embodiments. Accordingly, at 1102, the method 1100 may include receiving, using the communication device, at least one response from the at least one primary third device based on the transmitting of the referral data.

Further, at 1104, the method 1100 may include identifying, using the processing device, at least one facility of the at least one primary second facility.

Further, at 1106, the method 1100 may include generating, using the processing device, a notification based on the identifying of the at least one facility.

Further, at 1108, the method 1100 may include transmitting, using the communication device, the notification to at least one of the at least one second device, the at least one first device, and at least one device of the at least one primary third device. Further, the at least one device may be associated with the at least one facility.

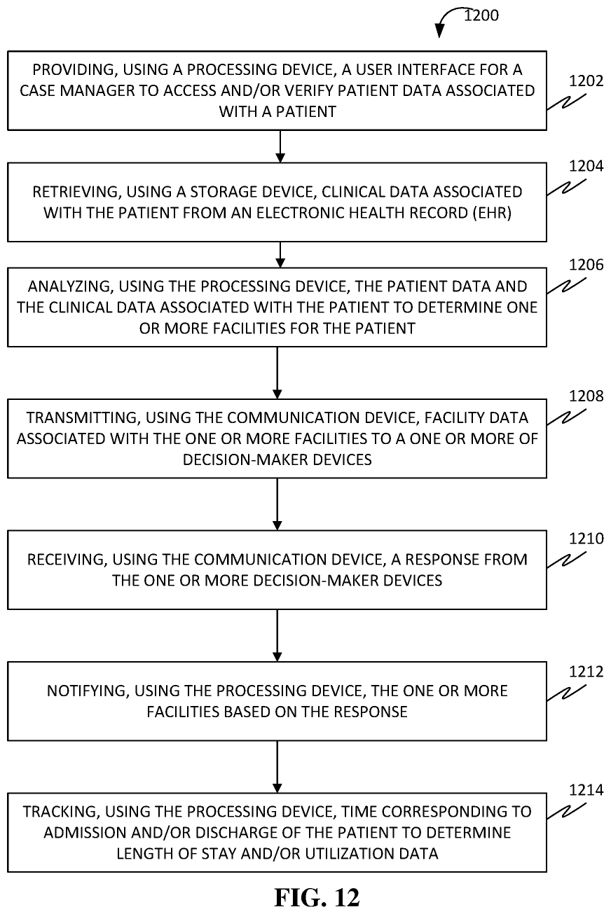

FIG. 12 is a flowchart of a method 1200 to facilitate hospital dispatch planning, in accordance with some embodiments. Accordingly, at 1202, the method 1200 may include a step of providing, using a processing device, a user interface (corresponding to a case manager application) for a case manager to access and/or verify patient data associated with a patient. Further, the patient, in an instance, may be an individual that may be treated in a hospital/clinic, and/or who may need some sort of post-acute care placement. Further, the case manager, in an instance, may be an individual that may be responsible for verifying the patient data associated with the patient. Further, the patient data, in an instance, may be data that may reflect any medical and/or non-medical information about the patient. For instance, the patient data may include, but not limited to, patient's identity information (e.g. patient's ID number issued by a hospital/clinic), insurance information (e.g. primary insurance information, secondary insurance information, preauthorization information, etc.), social information (encoding, mental representation, response accessing, evaluation, enactment, etc.) and so on. Further, the case manager, in an instance, may verify the patient data by interacting with the user interface on a case manager device. Further, the case manager device, in an instance, may be any user device that may be operated by the case manager associated with the hospital/clinic. Further, the case manager device, in an instance, may be configured to provide the user interface for the case manager to communicate with the online platform 100. Further, the case manager device, in an instance, may include IoT based devices such as (but not limited to) a smartphone, a smartwatch, a PC, a laptop, and so on. Further, the user interface (corresponding to the case manager application), in an instance, may allow the case manager to select hospitals and/or clinics in order to retrieve patient data from a database (such as Electronic health record) corresponding to that hospital and/or clinic. For instance, the user interface corresponding to the case manager application may allow the case manager to select a hospital (e.g. “Mercy Hospital”) in order to retrieve a list of patients corresponding to that hospital. For instance, the case manager may select “all patients” section from the user interface which may provide a list of patients. Further, the user interface, in an instance, may allow the case manager to select a patient (from the list of patients) to view and/or verify patient data associated with the patient corresponding to the hospital and/or clinic. For instance, the case manager may select “verify” button on the user interface in order to indicate that the patient data for the patient (such as a patient named “Shirai Subaru” have been verified.

Further, at 1204, the method 1200 may include a step of retrieving, using a storage device, clinical data associated with the patient from an Electronic Health Record (EHR). Accordingly, once the patient data may be verified by the case manager through the user interface, the online platform 100 may be configured to retrieve the clinical data associated with the patient from the Electronic Health Record (EHR). Further, the clinical data, in an instance, may be medical data that may reflect historic and/or present medical condition associated with the patient. For instance, the medical data may include, but not limited to, Physician progress notes and Speech Therapy, Occupational Therapy and Physical Therapy progress notes, and so on. Further, in another instance, the clinical data may include information contained within the progress notes of treating physicians, physical therapists, occupational therapists, and/or speech therapists. Further, the Electronic Health Record (EHR), in an instance, may be a database (such as databases 114) that may be configured to store clinical data and/or patient data in an organized form that may be accessed electronically by the online platform 100. For instance, the EHR may be configured to store a wide range of information such as (but not limited to) demographics, medical history, medication and allergies, immunization status, laboratory test results, radiology images, vital signs, personal statistics like age and weight, and billing information, and so on. Further, in some embodiments, the online platform 100 may be configured to retrieve clinical data from external databases other than EHR.

Further, at 1206, the method 1200 may include a step of analyzing, using the processing device, the patient data, and the clinical data associated with the patient to determine one or more facilities for the patient. Accordingly, the online platform 100, in an instance, may be configured to process the patient data and/or the clinical data associated with the patient to determine the one or more facilities that may be optimal for the patient as per the patient's need. Further, the one or more facilities, in an instance, may be post-acute care facilities that may be configured for providing necessary medical amenities to the patient. Further, the one or more facilities, in an instance, may be optimal for the patient based on the patient data and/or the clinical data associated with the patient. For instance, the one or more facilities may be optimal for the patient when the one or more facilities may meet the patient's needed level of acuity (such as level of services required depending on how sick the patient is and/or how much assistance may be needed). Further, to determine the patient's needed level of acuity, the online platform 100 may perform processes such as (but not limited to) data extraction (e.g. numerical value extraction, format conversion, etc.), data analysis (e.g. statistical analysis, etc.), image analysis (e.g. OCR, object recognition, face recognition, etc.), audio analysis (e.g. speech recognition, speaker recognition, etc.) and so on for the patient data and/or the clinical data associated with the patient. Further, in some embodiments, the online platform 100 may be configured to analyze data associated with the post-acute care facilities (e.g. data reflecting services provided by the one or more facilities, location of the one or more facilities, type of the one or more facilities, and so on) and/or perform matching algorithm to determine the one or more facilities that may be optimal for the patient based on the patient's need. Further, the matching algorithm, in an instance, may be based on a Boolean decision tree algorithm with Diagnosis-related group (DRG) specific criteria that may improve in matching the one or more facilities with the patient's need more accurately. Further, in some instances, the online platform 100 may perform OCR on the clinical data (such as progress notes from a physician associated with the patient) to determine if the patient may require any specific aid (e.g. walking aid), if yes, then the online platform 100 may identify the one or more facilities which may be capable of providing specific assistance (e.g. walking aid) for the patient. Further, in another instance, the online platform 100 may perform image analysis on the clinical data (such as radiological images) associated with the patient to determine the one or more facilities that may provide the required assistance to the patient as per the clinical data of the patient.

Further, at 1208, the method 1200 may include a step of transmitting, using the communication device, facility data associated with the one or more facilities to one or more of decision-maker devices. Accordingly, the facility data, in an instance, may be any data and/or information about the one or more facilities which may be of something of interest for a decision-maker to decide which facility (out of the one or more facilities) may be ideal for the patient. For instance, the facility data may include information such as, but not limited to, multimedia content (e.g. images, videos, and so on associated with the one or more facilities), contact information (e.g. phone number, email address, fax number, etc. associated with the one or more facilities), location information (e.g. address, GPS coordinates, etc. associated with the one or more facilities), and/or other information (e.g. services provided, fee structure, type of facility and so on associated with the one or more facilities), etc. Further, the online platform 100, in an instance, may be configured to transmit the facility data to the one or more decision-maker devices through a wireless transmitter. The wireless transmitter, in an instance, may transmit the facility data over, but not limited to, a Wi-Fi, a Bluetooth™ an electromagnetic waveform, ultra-sound, cellular (5G) and/or an Infra-red, etc. Further, the one or more decision-maker devices, in an instance, may include user devices that may be operated by the decision maker. Further, the one or more decision-maker devices, in an instance, may include IoT based devices such as (but not limited to) a smartphone, a smartwatch, a PC, a laptop, and so on. Further, the decision maker, in an instance, may be patients and/or other individuals that may be directly or indirectly linked with the patient, such as caretakers, family members, and so on, who may make decisions for the patient. Further, the one or more decision-maker devices, in an instance, may be configured to provide a user interface corresponding to a decision maker application that may allow the decision maker to select and/or view appropriate match with the facility data associated with the one or more facilities, so that the decision maker may make an informed choice of the facility for the patient. For instance, the user interface corresponding to the decision maker application may allow the decision maker to view a list of facilities with images and names (such as “Hawthorne Village,” “Pitie-salpetriere Hospital” etc.). Further, the user interface, in an instance, may allow the decision maker to view real-time information on the availability of the one or more facilities. For instance, the user interface may allow the decision maker to view if the one or more facilities may be vacant for the patient and/or when the one or more facilities may be available for the patient. Further, the user interface, in an instance, may allow the decision maker to plan and/or schedule a visit to the one or more facilities if required.

Further, at 1210, the method 1200 may include a step of receiving, using the communication device, a response from the one or more decision-maker devices.

Accordingly, the response, in an instance, may be any reply from the decision maker with regard to selecting the one or more facilities for the patient. Further, in some embodiment, the response may be an action performed by the decision maker with regard to selecting the one or more facilities for the patient. Further the action, in an instance, may include providing an acceptance/rejection for a post-acute care facility that may reflect an opinion of the decision maker. For instance, the decision maker (such as a caretaker) may ‘accept’ a facility out of the one or more facilities by clicking on ‘select facility’ that may be presented on the user interface corresponding to the decision maker application and/or may reflect a preference of the decision maker for the facility. Further, once a facility (such as a facility named “Hawthorne Village”) may be selected by the decision maker through the user interface, an indication such as a tick mark may be presented to the decision maker such as a tick-mark. Further, in some embodiments, the response may be in a textual form (such as a message) that may include suggestions, questions, and/or advice with regard to the one or more facilities on the user interface. Further, in some embodiments, the response may include audiovisual data from the decision maker (through the decision-maker device) with regard to the one or more facilities. For instance, the decision maker (e.g. a caretaker) may express an opinion about the services provided by the one or more facilities by providing a voice message and/or a textual message to the one or more facilities. Further, in some embodiments, the online platform 100 may be configured to start a timer as soon as the facility data associated with the one or more facilities were made available to the decision maker. After a set amount of time, the online platform 100 may indicate to the case manager that a decision has not been reached yet, and manual follow up may be needed. At this stage, the online platform 100 may provide contact information (such as phone number, e-mail, and so on) associated with the decision maker for quick access. After another predetermined time period elapses, a more urgent call for follow up may be made to the case manager. If the decision maker may be non-responsive or non-participating, the case managers may have the ability to make the selection themselves.

Further, at 1212, the method 1200 may include a step of notifying, using the processing device, the one or more facilities based on the response. Accordingly, once a post-acute care facility may be selected by the decision maker through the user interface corresponding to the decision maker application, the one or more facilities may be notified about the patient. For instance, a facility selected by the decision maker may be provided an alert (such as an SMS, an email, and so on) which may inform the facility about the patient. Further, in some embodiments, a treating physician (that may be associated with the treatment of the patient in the hospital/clinic) may be notified via SMS that the patient has been matched to a selected facility and a referral for the facility may be generated which may be sent to a facility agent application. Further, the facility agent application, in an instance, may be operated by a facility agent that may be associated with the one or more facilities to communicate with the online platform 100. Further, the facility agent may log in to the facility agent application to communicate with the online platform 100. Further, the facility agent application, in an instance, may be configured to provide a list of facilities along with patients for each facility. Further, the facility agent application, in an instance, may allow the facility agent to view information such as (but not limited to) patient name, room number, status, etc. corresponding to each facility from the list of facilities.

Further, at 1214, the method 1200 may include a step of tracking, using the processing device, time corresponding to admission and/or discharge of the patient to determine the length of stay and/or utilization data. Accordingly, the online platform 100, in an instance, may be configured to provide insights that may allow the case manager to facilitate discharge planning by getting updates on patient status in real-time instead of meeting daily with clinicians to get updates on patient status. Further, the insights, in an instance, may allow the case manager to monitor the length of stay for the patients in order to incentivize a shorter length of stay. Further, the insights, in an instance, may allow the case manager (and/or hospital administration) to handle case management labor costs to the hospital efficiently and/or help clinicians spend more time treating patients.

In further embodiments, the method 1200 may include contacting a transportation service automatically as soon as a patient is discharged. For example, the transportation service may include one or more of Lyft Health™, UberHealth™′ and Acuity Link™ Further, the patient's location and address of the destination facility may be shared with the transportation service. This will save time over the current process of making arrangements for transportation manually once discharge has been ordered.

Further, the present disclosure teaches a case manager application, a facility agent application, a facility dashboard application, in accordance with some embodiments. Further, the facility dashboard application showing a list of patients along with details such as name, region, chronic disease, date added, phone number, source hospital, etc. Further, the facility dashboard application may display details (such as name, photo, experience, number of patients, etc.) associated with facility agents. Further, the facility dashboard application allows the user to add a facility agent (e.g. register a user with the online platform 100 as a facility agent), by providing input sections for details such as name, address, street, phone number, hospitals, experience, email addresses, region, and/or profile image, etc.

Further, the present disclosure teaches a hospital dashboard application with the dashboard (such as bar graphs, line graphs, etc.), in accordance with some embodiments. Accordingly, the hospital dashboard application, in an instance, may allow a user (such as an administrator) associated with one or more of hospitals to view information associated with dashboards (such as the insights that may be updated regularly by the online platform 100), patients, and/or case managers, etc.

Further, the hospital dashboard application shows a list of patients along with details such as name, region, chronic disease, discharge time, phone number, length of stay, etc.

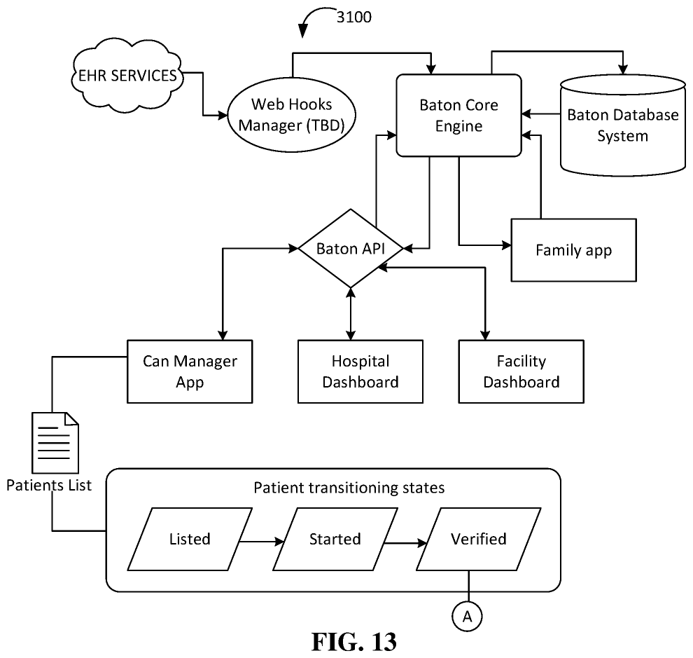

FIG. 13 shows an architecture overview 3100 associated with the system 200, in accordance with some embodiments. Accordingly, the architecture includes one or more of the case manager application, a hospital dashboard, and a facility dashboard, which may communicate with a Baton Core Engine via Baton API. Further, a family application may communicate with the Baton Core Engine. Further, the Baton Core Engine is connected to a Baton Database system. EHR services may be connected with the Baton Core Engine via WebHooks Manager.

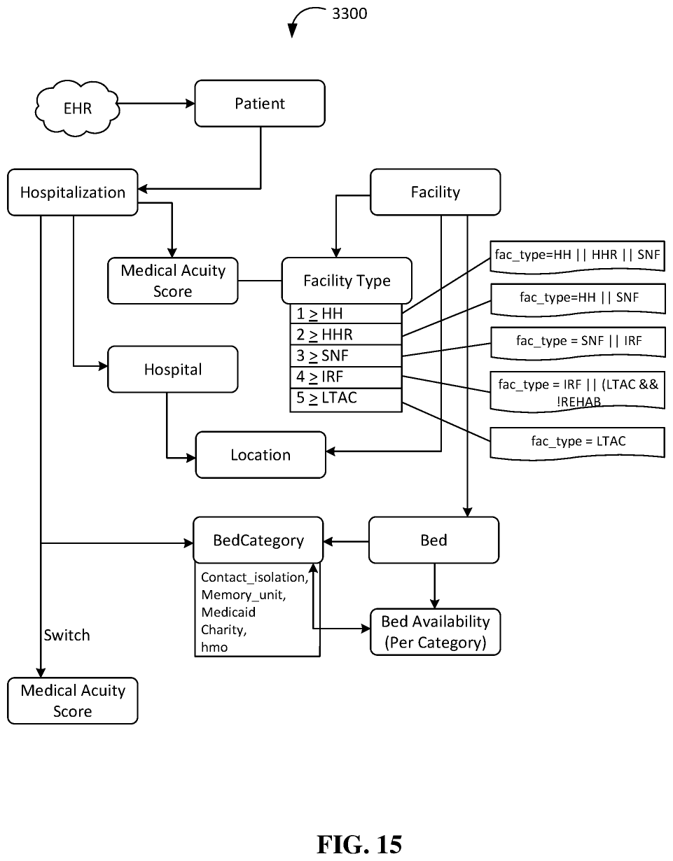

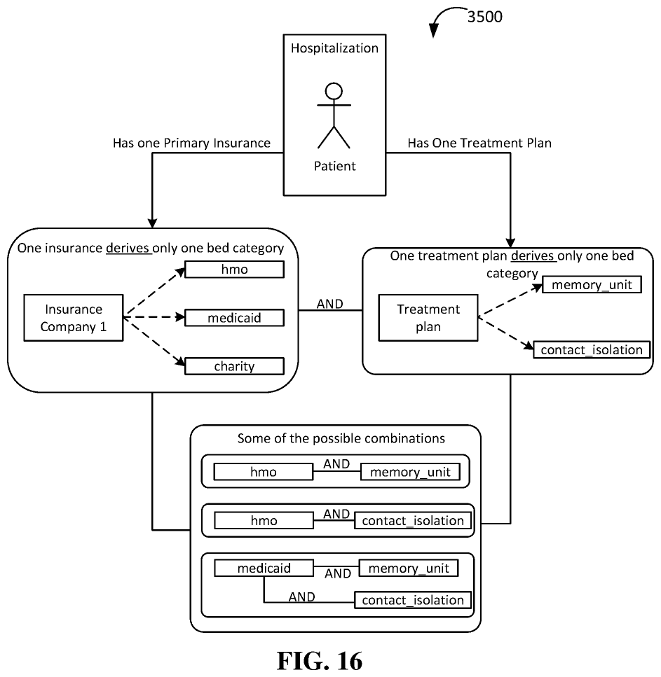

Further, the case manager application may provide a patient's list. The patients' status changes from listed to started to verified. Thereafter, a facility matching algorithm (explained in detail in FIG. 15-16) finds facilities matching a patient's requirements based on the patient's location, treatment plan, individual requirements, and custom conditions.

If, no matching facilities are found then the patient status goes to uncovered from verified. However, if matching facilities are found, then the patient status goes from verified to placed pending to discharged.

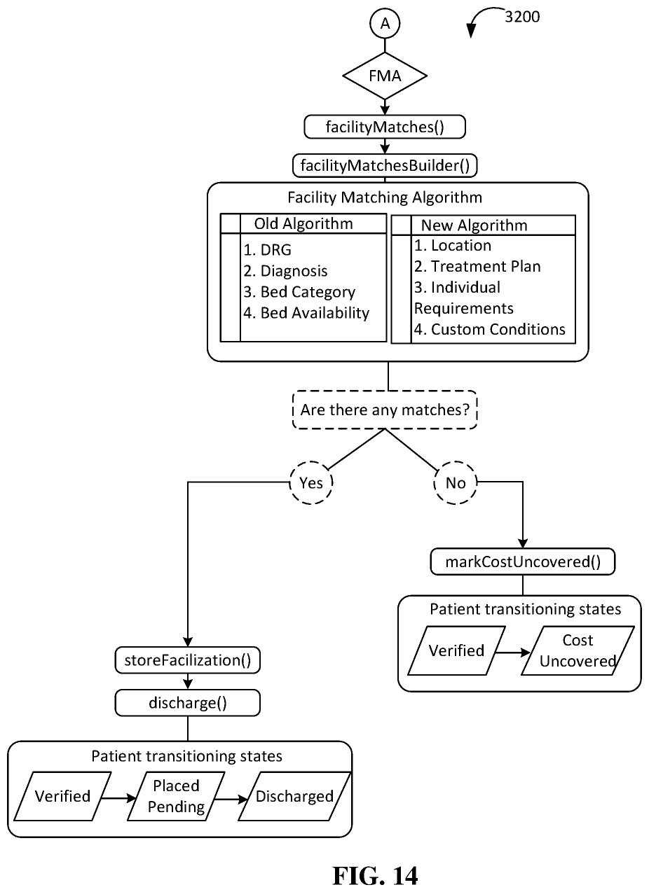

FIG. 14 shows the architecture overview 3200, in accordance with some embodiments.

FIG. 15 is a flow diagram of a method 3300 for facility matching, in accordance with some embodiments.

FIG. 16 is a flow diagram of a method 3500 for assigning patient bed categories, in accordance with some embodiments.

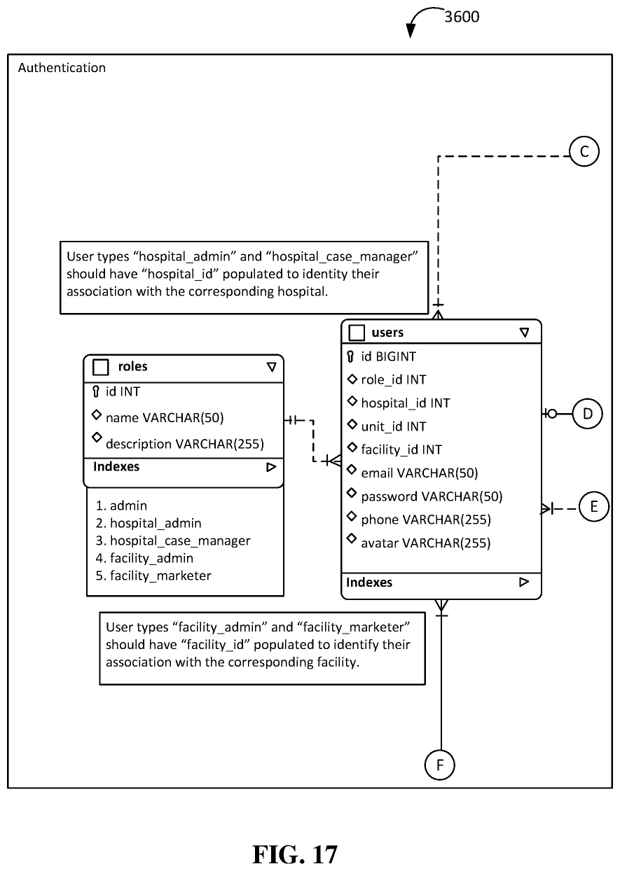

FIG. 17 shows a DataBase (DB) schema 3600 associated with the system 200, in accordance with some embodiments.

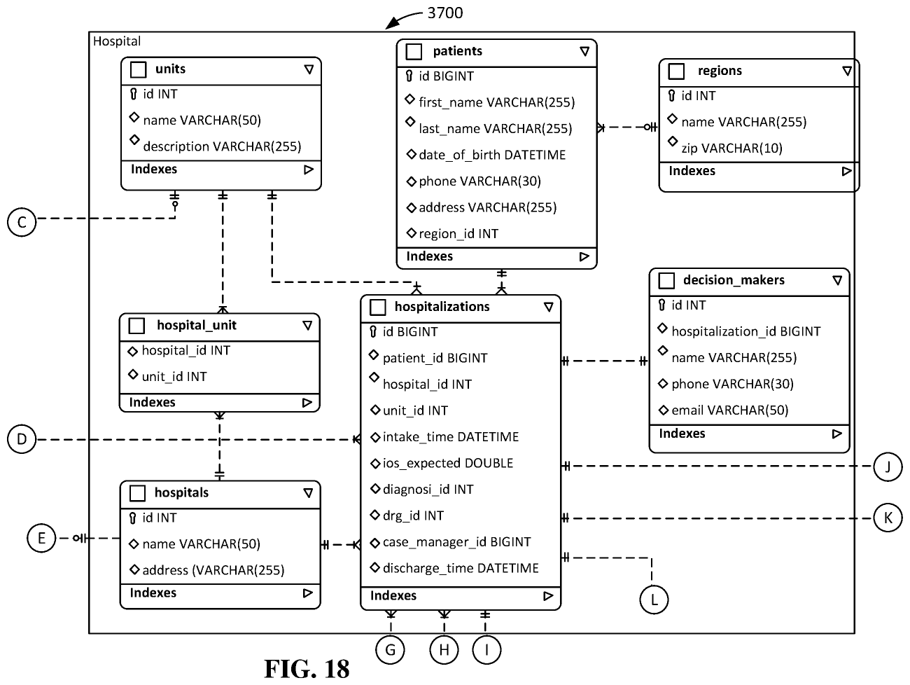

FIG. 18 shows a DB schema 3700 associated with the system 200, in accordance with some embodiments.

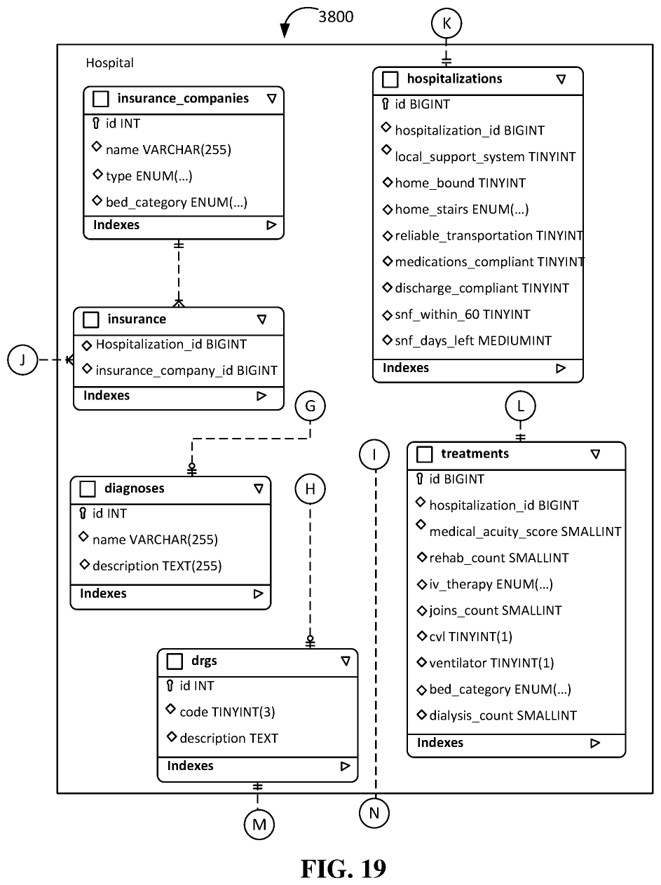

FIG. 19 shows a DB schema 3800 associated with the system 200, in accordance with some embodiments.

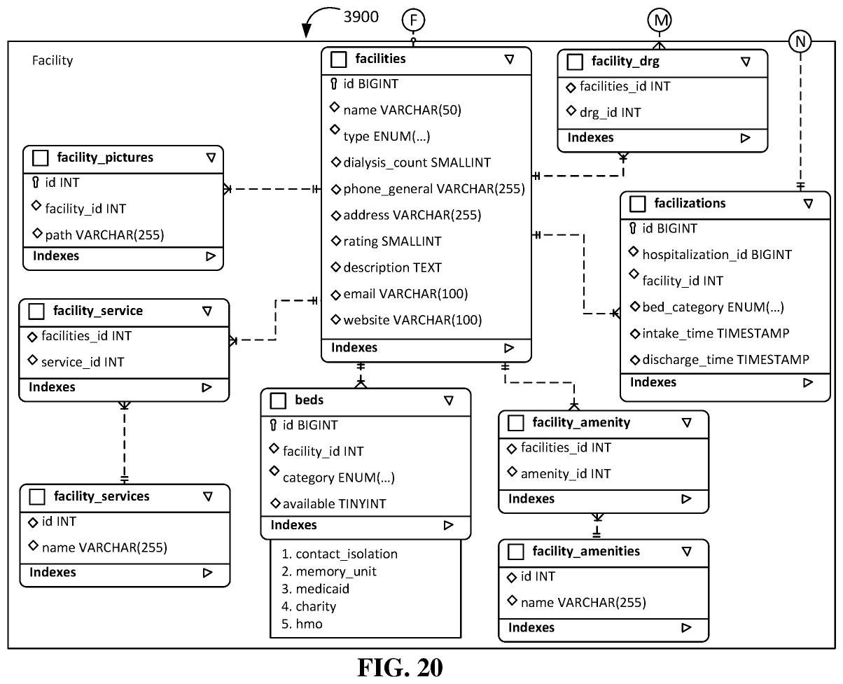

FIG. 20 shows the DB schema 3900 associated with the system 200, in accordance with some embodiments.

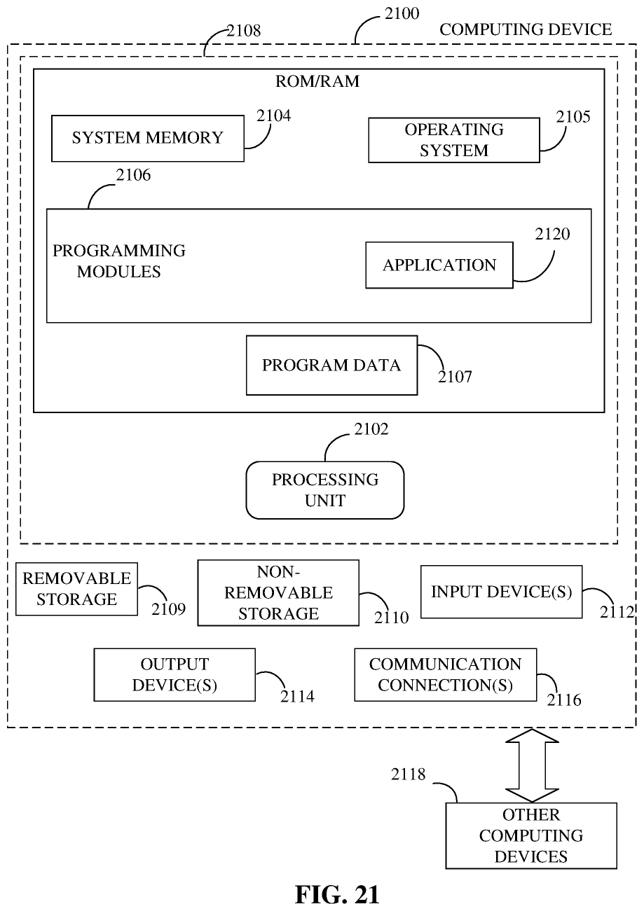

With reference to FIG. 21, a system consistent with an embodiment of the disclosure may include a computing device or cloud service, such as computing device 2100. In a basic configuration, computing device 2100 may include at least one processing unit 2102 and a system memory 2104. Depending on the configuration and type of computing device, system memory 2104 may comprise, but is not limited to, volatile (e.g. random-access memory (RAM)), non-volatile (e.g. read-only memory (ROM)), flash memory, or any combination. System memory 2104 may include operating system 2105, one or more programming modules 2106, and may include a program data 2107. Operating system 2105, for example, may be suitable for controlling computing device 2100's operation. In one embodiment, programming modules 2106 may include image-processing module, machine learning module. Furthermore, embodiments of the disclosure may be practiced in conjunction with a graphics library, other operating systems, or any other application program and is not limited to any particular application or system. This basic configuration is illustrated in FIG. 21 by those components within a dashed line 2108.

Computing device 2100 may have additional features or functionality. For example, computing device 2100 may also include additional data storage devices (removable and/or non-removable) such as, for example, magnetic disks, optical disks, or tape. Such additional storage is illustrated in FIG. 21 by a removable storage 2109 and a non-removable storage 2110. Computer storage media may include volatile and non-volatile, removable and non-removable media implemented in any method or technology for storage of information, such as computer-readable instructions, data structures, program modules, or other data. System memory 2104, removable storage 2109, and non-removable storage 2110 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2100. Any such computer storage media may be part of device 2100. Computing device 2100 may also have input device(s) 2112 such as a keyboard, a mouse, a pen, a sound input device, a touch input device, a location sensor, a camera, a biometric sensor, etc. Output device(s) 2114 such as a display, speakers, a printer, etc. may also be included. The aforementioned devices are examples and others may be used.

Computing device 2100 may also contain a communication connection 2116 that may allow device 2100 to communicate with other computing devices 2118, such as over a network in a distributed computing environment, for example, an intranet or the Internet. Communication connection 2116 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2104, including operating system 2105. While executing on processing unit 2102, programming modules 2106 (e.g., application 2120) may perform processes including, for example, one or more stages of methods, algorithms, systems, applications, servers, databases as described above. The aforementioned process is an example, and processing unit 2102 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A method for facilitating managing facilities for patients, the method comprising:
receiving, using a communication device, at least one identifier associated with a patient of at least one patient from at least one first device;
analyzing, using a processing device, the at least one identifier;
retrieving, using a storage device, clinical data and patient data associated with the patient from an electronic health record associated with at least one first facility based on the analyzing of the at least one identifier, wherein the patient is associated with the at least one first facility;
analyzing, using the processing device, the clinical data and the patient data;
determining, using the processing device, at least one patient characteristic associated with the patient based on the analyzing of the clinical data and the patient data;
identifying, using the processing device, at least one second facility based on the determining of the at least one patient characteristic;
transmitting, using the communication device, the at least one second facility to at least one second device;
initiating, using the processing device, a timer based on the transmitting of the at least one second facility, wherein the timer is configured for expiring after at least one predetermined duration;
generating, using the processing device, an indication corresponding to a response receivable from the at least one second device prior to the expiring of the timer;
determining, using the processing device, at least one primary second facility of the at least one second facility based on the generating of the indication;
generating, using the processing device, referral data of the patient based on the determining of the at least one primary second facility; and
transmitting, using the communication device, the referral data to at least one primary third device associated with the at least one primary second facility.

2. The method of claim 1, wherein the timer is configurable for pausing prior to the expiring based on the response, wherein the pausing of the timer prevents the expiring of the timer.

3. The method of claim 1 further comprising:
generating, using the processing device, at least one alert subsequent to the expiring of the timer; and
transmitting, using the communication device, the at least one alert to the at least one first device.

4. The method of claim 1 further comprising:
retrieving, using the storage device, at least one decision maker information associated with at least one decision maker subsequent to the expiring of the timer; and
transmitting, using the communication device, the at least one decision maker information to the at least one first device.

5. The method of claim 4 further comprising:
initiating, using the processing device, a first timer based on the transmitting of the at least one decision maker information, wherein the first timer is configured for expiring after at least one first predetermined duration;
transmitting, using the communication device, the at least one second facility to the at least one first device subsequent to the expiring of the first timer; and
receiving, using the communication device, at least one primary second facility indication corresponding to the at least one primary second facility from the at least one first device, wherein the determining of the at least one primary second facility of the at least one second facility is based on the receiving of the at least one primary second facility indication.

6. The method of claim 1 further comprising:
receiving, using the communication device, at least one second facility information from at least one third device;
analyzing, using the processing device, the at least one second facility information based on at least one predetermined criterion;
generating, using the processing device, at least one facility characteristic of each second facility of the at least one second facility based on the analyzing of the at least one second facility information; and
storing, using the storage device, the at least one facility characteristic and the at least one second facility information.

7. The method of claim 6 further comprising:
retrieving, using the storage device, the at least one facility characteristic associated with the each second facility; and
matching, using the processing device, the at least one patient characteristic with the at least one facility characteristic based on the retrieving of the at least one facility characteristic, wherein the identifying of the at least one primary second facility is based on the matching.

8. The method of claim 7, wherein each patient characteristic of the at least one patient characteristic comprises a predetermined value, wherein the method comprises:
identifying, using the processing device, at least one first patient characteristic of the at least one patient characteristic for the each second facility of the at least one second facility based on the matching; and
generating, using the processing device, a score of the each second facility based on the identifying of the at least one first patient characteristic, wherein the score comprises the predetermined value for each first patient characteristic of the at least one first patient characteristic, wherein the identifying of the at least one primary second facility is based on the generating of the score.

9. The method of claim 1 further comprising:
generating, using the processing device, at least one status of the patient based on the analyzing of the patient data and the clinical data, wherein the identifying of the at least one second facility is based on the generating of the at least one status; and
transmitting, using the communication device, the at least one status to the at least one first device.

10. The method of claim 1 further comprising:
receiving, using the communication device, at least one response from the at least one primary third device based on the transmitting of the referral data;
identifying, using the processing device, at least one facility of the at least one primary second facility;
generating, using the processing device, a notification based on the identifying of the at least one facility; and
transmitting, using the communication device, the notification to at least one of the at least one second device, the at least one first device, and at least one device of the at least one primary third device, wherein the at least one device is associated with the at least one facility.

11. A system for facilitating managing facilities for patients, the system comprising:
a communication device configured for:
receiving at least one identifier associated with a patient of at least one patient from at least one first device;
transmitting at least one second facility to at least one second device
transmitting referral data to at least one primary third device associated with at least one primary second facility;
a storage device configured for retrieving clinical data and patient data associated with the patient from an electronic health record associated with at least one first facility based on the analyzing of the at least one identifier, wherein the patient is associated with the at least one first facility; and
a processing device communicatively coupled with the communication device and the storage device, wherein the processing device is configured for:
analyzing the at least one identifier;
analyzing the clinical data and the patient data;
determining at least one patient characteristic associated with the patient based on the analyzing of the clinical data and the patient data;
identifying the at least one second facility based on the determining of the at least one patient characteristic;
initiating a timer based on the transmitting of the at least one second facility, wherein the timer is configured for expiring after at least one predetermined duration;
generating an indication corresponding to a response receivable from the at least one second device prior to the expiring of the timer;
determining the at least one primary second facility of the at least one second facility based on the generating of the indication; and
generating the referral data of the patient based on the determining of the at least one primary second facility.

12. The system of claim 11, wherein the timer is configurable for pausing prior to the expiring based on the response, wherein the pausing of the timer prevents the expiring of the timer.

13. The system of claim 11, wherein the processing device is further configured for generating at least one alert subsequent to the expiring of the timer, wherein the communication device is further configured for transmitting the at least one alert to the at least one first device.

14. The system of claim 11, wherein the storage device is further configured for retrieving at least one decision maker information associated with at least one decision maker subsequent to the expiring of the timer, wherein the communication device is further configured for transmitting the at least one decision maker information to the at least one first device.

15. The system claim 14, wherein the processing device is further configured for initiating a first timer based on the transmitting of the at least one decision maker information, wherein the first timer is configured for expiring after at least one first predetermined duration, wherein the communication device is further configured for:
transmitting the at least one second facility to the at least one first device subsequent to the expiring of the first timer; and
receiving at least one primary second facility indication corresponding to the at least one primary second facility from the at least one first device, wherein the determining of the at least one primary second facility of the at least one second facility is based on the receiving of the at least one primary second facility indication.

16. The system of claim 11, wherein the communication device is further configured for receiving at least one second facility information from at least one third device, wherein the processing device is further configured for:
analyzing the at least one second facility information based on at least one predetermined criterion; and
generating at least one facility characteristic of each second facility of the at least one second facility based on the analyzing of the at least one second facility information, wherein the storage device is further configured for storing the at least one facility characteristic and the at least one second facility information.

17. The system of claim 16, wherein the storage device is further configured for retrieving the at least one facility characteristic associated with the each second facility, wherein the processing device is further configured for matching the at least one patient characteristic with the at least one facility characteristic based on the retrieving of the at least one facility characteristic, wherein the identifying of the at least one primary second facility is based on the matching.

18. The system of claim 17, wherein each patient characteristic of the at least one patient characteristic comprises a predetermined value, wherein the processing device is further configured for:
identifying at least one first patient characteristic of the at least one patient characteristic for the each second facility of the at least one second facility based on the matching; and
generating a score of the each second facility based on the identifying of the at least one first patient characteristic, wherein the score comprises the predetermined value for each first patient characteristic of the at least one first patient characteristic, wherein the identifying of the at least one primary second facility is based on the generating of the score.

19. The system of claim 11, wherein the processing device is further configured for generating at least one status of the patient based on the analyzing of the patient data and the clinical data, wherein the identifying of the at least one second facility is based on the generating of the at least one status, wherein the communication device is further configured for transmitting the at least one status to the at least one first device.

20. The system of claim 11, wherein the communication device is further configured for:
receiving at least one response from the at least one primary third device based on the transmitting of the referral data; and
transmitting a notification to at least one of the at least one second device, the at least one first device, and at least one device of the at least one primary third device, wherein the at least one device is associated with at least one facility, wherein the processing device is further configured for:
identifying the at least one facility of the at least one primary second facility; and
generating the notification based on the identifying of the at least one facility.
